(19) United States
(12) Patent Application Publication
(10) Pub. No.: US 2023/0405266 A1
(43) Pub. Date: Dec. 21, 2023

(54) CATHETER WRAP

(71) Applicants: Tara HEAIRET, Spearfish, SD (US); Ann MOLLMAN, Spearfish, SD (US)

(72) Inventors: Tara HEAIRET, Spearfish, SD (US); Ann MOLLMAN, Spearfish, SD (US)

(21) Appl. No.: 18/210,880

(22) Filed: Jun. 16, 2023

Related U.S. Application Data
(60) Provisional application No. 63/352,931, filed on Jun. 16, 2022.

(51) Int. Cl. A61M 25/00; B08B 9/023

(52) U.S. Cl. CPC A61M 25/002; B08B 2209/02; A61M 2025/0019; B08B 9/023

(57) ABSTRACT

A cleansing catheter wrap kit includes a sterile package, a hollow enclosure disposed in the sterile package, and a cleansing solution in the sterile package. The hollow enclosure has a central channel and a slit in an exterior surface extending at least partially along a length of the hollow enclosure. The hollow enclosure is formed of a resilient material, and the slit is biased toward a closed position. The resilient material of the hollow enclosure is absorbent to receive and hold the cleansing solution. In use, the central channel is positioned over the catheter end, and the catheter end is scrubbed with the cleansing solution in the hollow enclosure.

### CROSS-REFERENCES TO RELATED APPLICATIONS

This application claims the benefit of U.S. Provisional Patent Application No. 63/352,931, filed Jun. 16, 2022, the entire content of which is herein incorporated by reference.

### STATEMENT REGARDING FEDERALLY SPONSORED RESEARCH OR DEVELOPMENT

(NOT APPLICABLE)

### BACKGROUND

The invention relates to a sterile wrap for a catheter and, more particularly, to a pre-soaked cleansing, wrap for catheters that is easily utilized and secured.

Currently, catheters used for dialysis, central lines, IVs, etc. must be cleansed before and after use with a suitable solution such as Betadine. Medical professionals typically wrap the catheter ends with a cloth material soaked in the cleaning solution and sometimes even wrap the catheter end and soaked cloth in a gauze material. The gauze material is intended to cover the soaked cleansing wrap and secure the cleansing wrap in place until it is time to connect/disconnect the catheter, typically around a minute.

Oftentimes, however, if the patient moves a certain way or just due to the resiliency of the gauze wrap, the gauze wrap can fall off, after which the cleansing wrap also tends to fall off. The medical professional consequently does not know whether the catheter is suitably clean for use.

### SUMMARY

An object of the described embodiments is to provide a catheter wrap/cleanser assembly or kit that is easy to use/install and if need be will stay in place once fitted. The described catheter wrap will decrease infection, morbidity and mortality rates while also improving staff efficiencies.

In an exemplary embodiment, a cleansing catheter wrap kit includes a sterile package, a hollow enclosure disposed in the sterile package, and a cleansing solution disposed in the sterile package. The hollow enclosure includes a central channel and a slit in an exterior surface extending at least partially along a length of the hollow enclosure. The hollow enclosure is formed of a resilient material, and the slit is biased toward a closed position. The resilient material of the hollow enclosure is absorbent to receive and hold the cleansing solution.

The sterile package may include a perforated tear-off closure. The resilient material may be soaked in the cleansing solution in the sterile package. The hollow enclosure may be a cylindrical tube. In this context, the slit may extend along an entirety of the hollow enclosure length. The cleansing solution may include one of an antimicrobial, antiseptic, alcohol, and/or betadine solution. The central channel may include bumps or fingerling projections. At least one end of the hollow enclosure may include an interior lip. The resilient material may be one of a porous sponge and foam. In this context, the resilient material may be at least of one of synthetic, woven, non-woven, and knitted.

In another exemplary embodiment, a method of cleansing and wrapping a catheter end with the cleansing catheter wrap kit of the described embodiments includes the steps of (a) opening the sterile package and removing the hollow enclosure; (b) positioning the central channel around the catheter end; and (c) scrubbing the catheter end with the hollow enclosure.

Opening the sterile package may include tearing a perforated tear-off closure. Step (b) may be practiced by opening the slit to expose the central channel and placing the central channel around the catheter end. Step (c) may be practiced by rolling the central channel back and forth around the catheter end. The method may further include, after step (c), wrapping the hollow enclosure in gauze (e.g., to keep in place until ready to disconnect/reconnect the catheters).

### BRIEF DESCRIPTION OF THE DRAWINGS

These and other aspects and advantages will be described in detail with reference to the accompanying drawings, in which:

FIG. 1 is a perspective view of a hollow enclosure used with the cleansing catheter wrap of the described embodiments;

FIG. 2 is a cross-sectional view through the central channel of the hollow enclosure;

FIG. 3 shows the hollow enclosure disposed in a sterile package; and

FIGS. 4-6 illustrate the process for cleansing and wrapping a catheter end with the cleansing catheter wrap of the described embodiments.

### DETAILED DESCRIPTION

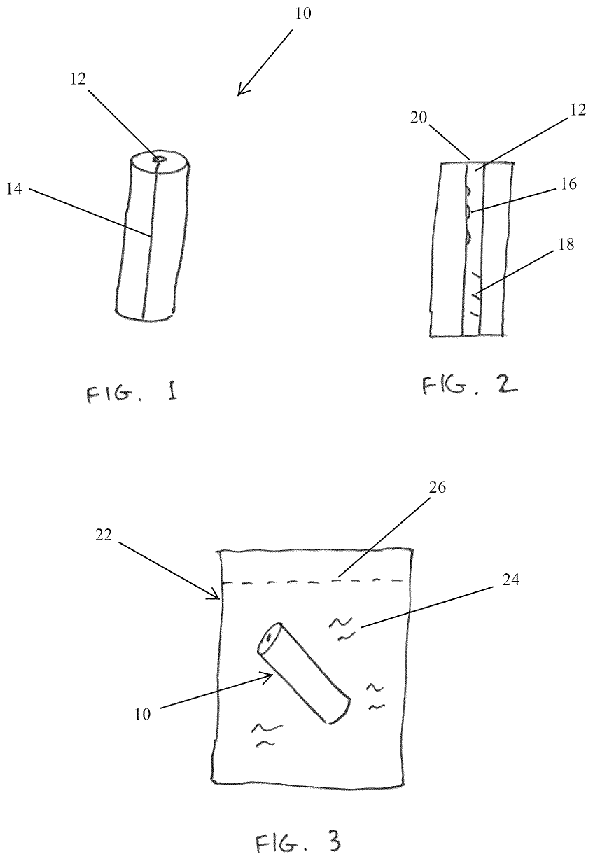

With reference to FIGS. 1-3, the cleansing catheter wrap assembly or wrap kit according to the described embodiments utilizes a hollow enclosure 10 with a central channel 12 and a slit 14 in an exterior surface extending at least partially along a length of the hollow enclosure 10. In some embodiments, the hollow enclosure 10 is in the form of a cylindrical tube. Other shapes and configurations may be utilized. The slit 14 may extend along an entirety of the hollow enclosure length.

With reference to the cross-section of the hollow enclosure in FIG. 2, the central channel 12 may be provided with bumps 16 and/or fingerling projections 18 to increase contact surfaces for the cleansing material to the catheter. At least one end of the hollow enclosure 10 may be provided with a lip 20 on the interior to hold the cleansing solution in place.

The hollow enclosure 10 can be provided in varying sizes, for example 1-2.5 inches long with a single slit 14 going from top to bottom on one side. The hollow enclosure 10 is preferably constructed of a resilient material so that the slit 14 is biased toward a closed position. The resilient material can flatten and regain its shape in use. The material may be made of a porous sponge, foam or other material that can absorb and transfer the cleansing solution. The material may be synthetic, woven, non-woven, knitted, etc.

The assembly or kit also includes a sterile package 22 in which the hollow enclosure 10 is disposed. In some embodiments, the sterile package 22 includes a perforated tear-off closure 26. The tear-off closure 26 facilitates opening the package 22. A cleansing solution 24 is also disposed in the sterile package 22. The resilient material of the hollow enclosure 10 is absorbent to receive and hold the cleansing solution 24. In use, the resilient material is soaked in the cleansing solution 24 in the sterile package 22.

The cleansing solution can be any suitable material for the intended purpose including, for example, an antimicrobial, antiseptic, alcohol, betadine or other cleansing solution.

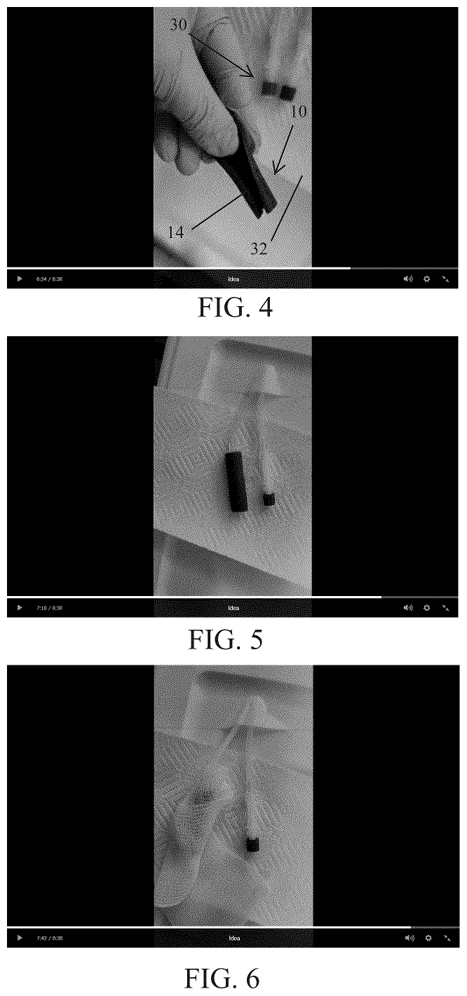

With reference to FIGS. 4-6, the catheter wrap assembly/kit is utilized for cleansing and wrapping a catheter end or connection port 30. In use, a drape 32 may first be placed between the patient and the catheter ends 30. The soaked hollow enclosure 10 is removed from the sterile package 22 after opening the sterile package 22. The central channel 12 is positioned over the catheter end 30. Due to the resiliency of the hollow enclosure 10, the slit 14 is naturally biased to close around the catheter end 30. Once placed, a medical professional can massage the soaked hollow enclosure 10 on the catheter end 30 to spread/apply the cleansing solution. The scrubbing of the catheter end 30 may be practiced by rolling the catheter end 30 back and forth inside the central channel 12, i.e., rolling the central channel 12 back and forth around the catheter end 30. In some instances, the hollow enclosure 10 may be left in place until the connection port is ready to be utilized by either connecting to another sterile port or disconnecting two ports.

In some embodiments, with the hollow enclosure 10 secured over the catheter end 30, the hollow enclosure is subsequently wrapped in gauze as shown in FIG. 6.

The cleansing catheter wrap of the described embodiments is easily fitted and secure once in place. The resilient hollow enclosure maintains a secure connection on the catheter ends. The described catheter wrap will decrease infection, morbidity and mortality rates while also improving staff efficiencies.

While the invention has been described in connection with what is presently considered to be the most practical and preferred embodiments, it is to be understood that the invention is not to be limited to the disclosed embodiments, but on the contrary, is intended to cover various modifications and equivalent arrangements included within the spirit and scope of the appended claims.

1. A cleansing catheter wrap kit comprising:
a sterile package;
a hollow enclosure disposed in the sterile package, the hollow enclosure including a central channel and a slit in an exterior surface extending at least partially along a length of the hollow enclosure, wherein the hollow enclosure is formed of a resilient material, and wherein the slit is biased toward a closed position; and
a cleansing solution disposed in the sterile package,
wherein the resilient material of the hollow enclosure is absorbent to receive and hold the cleansing solution.

2. A cleansing catheter wrap kit according to claim 1, wherein the sterile package includes a perforated tear-off closure.

3. A cleansing catheter wrap kit according to claim 1, wherein the resilient material is soaked in the cleansing solution in the sterile package.

4. A cleansing catheter wrap kit according to claim 1, wherein the hollow enclosure comprises a cylindrical tube.

5. A cleansing catheter wrap kit according to claim 4, wherein the slit extends along an entirety of the hollow enclosure length.

6. A cleansing catheter wrap kit according to claim 1, wherein the cleansing solution comprises one of an antimicrobial, antiseptic, alcohol, and betadine solution.

7. A cleansing catheter wrap kit according to claim 1, wherein the central channel comprises bumps.

8. A cleansing catheter wrap kit according to claim 1, wherein the central channel comprises fingerling projections.

9. A cleansing catheter wrap kit according to claim 1, wherein at least one end of the hollow enclosure comprises an interior lip.

10. A cleansing catheter wrap kit according to claim 1, wherein the resilient material comprises one of a porous sponge and foam.

11. A cleansing catheter wrap kit according to claim 10, wherein the resilient material is at least of one of synthetic, woven, non-woven, and knitted.

12. A method of cleansing and wrapping a catheter end with the cleansing catheter wrap kit of claim 1, the method comprising:
(a) opening the sterile package and removing the hollow enclosure;
(b) positioning the central channel around the catheter end; and
(c) scrubbing the catheter end with the hollow enclosure.

13. A method according to claim 12, wherein opening the sterile package comprises tearing a perforated tear-off closure.

14. A method according to claim 12, wherein step (b) is practiced by opening the slit to expose the central channel and placing the central channel around the catheter end.

15. A method according to claim 12, wherein step (c) is practiced by rolling the central channel back and forth around the catheter end.

16. A method according to claim 12, further comprising, after step (c), wrapping the hollow enclosure in gauze.

17. A method of cleansing and wrapping a catheter end with a cleansing catheter wrap kit including a sterile package, a hollow enclosure disposed in the sterile package, and a cleansing solution disposed in the sterile package, the method comprising:
(a) opening the sterile package and removing the hollow enclosure;
(b) positioning the central channel around the catheter end; and
(c) scrubbing the catheter end with the hollow enclosure.
